(19) United States
(12) Patent Application Publication
(10) Pub. No.: US 2026/0108236 A1
(43) Pub. Date: Apr. 23, 2026

(54) DEVICE FOR EYE POSITIONING AND IMMOBILIZATION DURING OPHTHALMOLOGICAL SURGERY

(71) Applicant: GABRIEL MORGADO SERVIÇOS MÉDICOS, LDA, Senhora da Hora (PT)

(72) Inventor: Gabriel José MORGADO SANTOS, Senhora da Hora (PT)

(21) Appl. No.: 19/488,192

(22) PCT Filed: Jun. 7, 2024
(86) PCT No.: PCT/IB2024/055611

§ 371 Date: Nov. 26, 2025

(30) Foreign Application Priority Data
Jun. 7, 2023 (PT) 118711
Aug. 16, 2023 (EP) 23191775.8

(51) Int. Cl. A61B 17/02

(52) U.S. Cl. CPC A61B 17/0231; A61F 9/00781

(57) ABSTRACT

Ophthalmic surgical device for eye positioning and immobilization using traction suture or sutures, comprising: a base configured for securing to a periocular region comprising the inferior periocular area and frontotemporal area, said base having an adhesive surface for adhering to the periocular region; wherein the base comprises an anchoring element for adjustable fixation of the traction suture or sutures; the base has a support edge for supporting, externally to the eyelids, against at least a part of a speculum-supported eyelid or against at least a part of an eyelid-retracting speculum. The anchoring element may comprise a recess for the suture or sutures to pass through and for holding said suture or sutures. The anchoring element may comprise a rotatable fastener for fastening the suture or sutures. The rotatable fastener may be rotatably coupled to the anchoring element.

### TECHNICAL FIELD

The present disclosure relates to an eye globe positioning and immobilization device for use during ophthalmological surgery, in particular during glaucoma, strabismus and pterygium surgery.

### BACKGROUND

When ophthalmological surgery procedures require eye positioning and immobilization, there is a need for effective, safe, practical means for positioning and immobilizing the eye during said procedures.

Examples of immobilization devices used for cornea refractive surgery and intravitreal injections are in the following documents.

Document EP1147754A1 discloses a suction ring which immobilizes and imparts vacuum pressure to the eyeball through an orifice, the vacuum pressure being distributed simultaneously from the ring through a peripheral channel situated in the same.

As another example, document CN112515846A discloses an intraocular injection auxiliary device.

In glaucoma surgery (trabeculectomy, deep sclerectomy, tube shunt devices, or other glaucoma devices) there is a need for eye positioning to expose a surface area of sclera, sub-tenon's space, periocular space, where surgeries are performed or devices are implanted.

The same need is necessary for strabismus and pterygium surgery, for example. Current methods are traction sutures that pass through the cornea or rectus muscles, that are secured by tightening a forceps to periocular drapes or using tape. These methods are not as effective, safe, and practical as ophthalmological surgeries require. Sutures fixation often slide, surgical area exposition is often limited, there is no adjustability and often there is need of human hand to help circumvent these limitations. There is thus a need for a device that addresses these problems.

One of the problems to be solved may be seen as the need in ophthalmic surgery for eye positioning, eye immobilization and eye position adjustability, in order to expose areas of the eye where certain surgeries are performed, such as areas behind the limbus, which are only exposed when the eye is positioned in extreme gaze positions. This is a problem that has more than 60 years. The need for its resolution has steadily increased as the number and different types of ophthalmic surgeries have also increased. To perform these surgeries, two conditions must be met:
1. The eyelids must be spread apart, to give access to the eye, which is efficiently accomplished with an eyelid speculum/retractor with two opposite arms and a pivotal point. This gives access to the eye. There are several types of this devices. The eye remains in primary position, this is with the gaze straight ahead such as in devices U.S. Pat. Nos. 7,985,180B2, 5,341,798A, CN202714969U, this exposure of the eye is sufficient to perform ocular surgeries that occur anterior to the corneal limbus such as cataract and refractive surgery, but insufficient to perform surgeries that occur posterior to the limbus. In some cases additional features were developed on these devices, US20070179346A1 describes an eyelid speculum with an aspiration system to avoid fluid accumulation in the conjunctiva fornix and WO2020080792A1 has a complex system for adjusting the thread on adjustable strabismus surgery. In the prior art, the most efficient devices to spread apart the eyelids are eyelid specula, attempts were performed with other devices, such as devices that were mainly designed as wound retractors, such as EP0156218A1. Its absence of efficacy and even danger to the delicate structures of the eye and adnexa, rendered them unusable in the field of ophthalmology, even for retracting eyelids. For clarity purposes devices destined to spread apart/retract the eyelids are not the scope of this invention, such as the previous referred ones. ;
2. To perform eye surgeries in ocular areas behind the limbus, the eye must be forced, in/to extreme gaze positions, to expose these areas, with traction sutures such as in the present disclosure.

The present disclosure does not pertain to an eyelid retractor, i.e. devices intended for the mobilization or separation of the eyelids. Instead, it pertains to the field of eye (eye globe, eyeball) positioning, eye immobilization, and eye position adjustability, in order to expose areas of the eye where certain eye surgeries are performed. The claimed device is directed to the eye globe itself, for rotating, positioning it and immobilizing it in a desired position.

The disclosure is thus directed to the second point, as it creates a solid anchor point for the eye traction sutures to be secured (avoiding suture slippage that commonly occurs in the state of art practice that is using tape or a forceps to secure the sutures), it allows adjustability of the eye position, enhancing gaze positions, that results in a broader exposed surface of the eye, where the surgeries can be performed.

Prior art devices are mostly directed to the eyelids, not the eye itself, and they cannot in any realistic way work on the second condition, this is positioning the eye in/to extreme gaze positions, and expose areas where surgeries are performed, that is the subject matter of the present disclosure.

These facts are disclosed to illustrate the technical problem addressed by the present disclosure.

### GENERAL DESCRIPTION

The present disclosure relates to an eye positioning and immobilization device for use during ophthalmological surgery, in particular during glaucoma surgery, pterygium or strabismus surgery. The device offers significant improvements ensuring that the eye is securely positioned and immobilized during surgery, allowing surgeons to perform procedures with greater precision and control. Eye and eye globe are used interchangeably in the present disclosure.

Advantages of the device of the present disclosure include that the device is safer, more practical and highly effective at eye positioning and immobilization during ophthalmological surgery. It allows for more surface area to be exposed for surgery, fine tuning of the positioning of the eye, adjustability and reversibility of direction of the positioning.

The disclosure is thus directed to creating a solid anchor point for an eye traction suture or sutures, namely as to point 2, referred above. The disclosure avoids suture slippage that commonly occurs in the state of art practice that is using tape or a forceps to secure the sutures; allows adjustability of the eye position by means of rotating a fastener or by means where a suture can be twisted; and allows the directing of the suture or sutures in order to provide vector forces on the eye, through for example lateral tabs, for enhancing gaze positions; thus resulting in a broader exposed surface of the eye, in precise exposition and positioning of the surgical area, where the surgeries can be performed. This device is normally to be used in conjunction with one of a multitude of eyelid specula, that accomplishes point 1, referred above, in the most effective way.

It is disclosed an ophthalmic surgical device for eye positioning and immobilization using traction suture or sutures, comprising:
a base configured for securing to a periocular region, directly or to surgical drapes covering that same area, wherein the periocular region comprises the inferior periocular area and frontotemporal area, said base having an adhesive surface for adhering to the periocular region;
wherein the base comprises an anchoring element for adjustable fixation [i.e. the fixation is variable and reversible so that adjustment can be provided] of the traction suture or sutures. Alternatively, the adhesive surface can be non-adhesive and the adhesive may be applied at a later time before securing the base at the periocular region.

In the disclosed ophthalmic surgical device, the base may have a support edge for supporting [i.e. abutting], externally to the eyelids, against at least a part of a speculum-supported eyelid or against at least a part of an eyelid speculum, such that the eyelid supported by the speculum, or the speculum itself, act as a stopper. The edge must be sufficiently thin, i.e. of a limited thickness, for engaging and supporting against at least a part of a speculum-supported eyelid or against at least a part of an eyelid speculum.

In the disclosed ophthalmic surgical device, the anchoring element is preferably configured for receiving the eye globe traction suture or sutures when the device is adhered to a periocular region by the adhesive surface.

In the disclosed ophthalmic surgical device, the anchoring element may be located in the adhesive surface region on an opposite surface of the base relative to the adhesive surface for securing the base to the periocular region for positioning and immobilization of the eye globe using the traction suture or suture. In particular, the anchoring element may be located directly opposite, relative to the base, to a region comprised in the adhesive surface.

It is disclosed an ophthalmic surgical device for eye globe positioning and immobilization using eye globe traction suture or sutures, comprising:
a base configured for securing to a periocular region comprising the inferior periocular and frontotemporal area, said base having an adhesive surface for adhering to the periocular region;
wherein the base comprises an anchoring element for adjustable fixation of the traction suture or sutures;
wherein the anchoring element is configured for receiving the eye globe traction suture or sutures when the device is adhered to a periocular region by the adhesive surface;
wherein the base has a support edge for supporting, externally to the eyelids, against at least a part of a speculum-supported eyelid or against at least a part of an eyelid speculum; and
wherein the anchoring element is located directly opposite, relative to the base, to a region comprised in the adhesive surface.

In an embodiment, the anchoring element comprises a recess [for example, a slot or a slit] for the suture or sutures to pass through and for holding said suture or sutures.

In an embodiment, the anchoring element comprises a rotatable fastener for fastening the suture or sutures; the rotatable fastener being preferably detachable from the anchoring element.

In an embodiment, the rotatable fastener is rotatably coupled to the anchoring element [i.e. the anchoring element serves as a rotation axle of the fastener].

In an embodiment, the attachment of the rotatable fastener and the anchoring element, has a shape for enhancing grasp by pushing the rotatable fastener towards the base, in particular the anchoring element tapering away from the base, for example the anchoring element being conical, for increasing grip when pushing the rotatable fastener towards the base, which results in a progressive and secure tightening of the eye traction suture, that runs in between the rotatable fastener and anchoring element, in opposite directions, preventing its slippage.

In an embodiment, the rotatable fastener comprises a sulcus for the suture or sutures to wind around the rotatable fastener and for holding said suture or sutures.

In an embodiment, the rotatable fastener comprises one or more recesses (or sulcus) for the suture or sutures to pass through and for holding said suture or sutures, in particular the one or more recesses (or sulcus) being arranged on a surface of the rotatable fastener facing said base or facing outwards in respect of the rotatable fastener.

In an embodiment, the anchoring element comprises a detachable, for example clip-on, fastener for fastening the suture or sutures.

In the invention, the base has a support edge for supporting, i.e. abutting, externally to the eyelids, against at least a part of a speculum-supported eyelid or against at least a part of an eyelid speculum.

In an embodiment, the base further comprises two laterally protruding tabs arranged between the support edge and the anchoring element for guiding the one or more sutures, in particular for guiding the one or more sutures towards the orbital rim, further in particular for guiding the one or more sutures towards the inferior orbital rim, in the desired direction.

In an embodiment, the base further comprises a ridge for applying friction to the suture or sutures, and raising the suture or sutures from the base towards a suture running position in the anchoring element or the fastener, if existing.

An embodiment further comprises one or more protrusions between the support edge, including the support edge itself, and the anchoring element for deflecting the one or more sutures.

In an embodiment, the base comprises a plurality of said protrusions wherein the plurality of the protrusions is arranged along an axis between the support edge and the anchoring element.

In an embodiment, the base has a curvature, or curvature and counter curvature, for contouring adjacently the periocular area.

In an embodiment, the base is an elongated base having two ends.

In an embodiment, the base has a narrowing between first and second end.

In an embodiment, the base has two laterally protruding tabs arranged on the narrowed region of the base, or in between extremities of the base.

In an embodiment, the anchoring element is a protruding anchor.

In an embodiment, the anchoring element is an anchor with a hollow volume for passing through and anchoring the traction suture or sutures.

In an embodiment, the rotatable fastener is a rotatable knob, rotatable torniquet or a rotatable screw cap.

In an embodiment, the rotatable fastener comprises one or more levers for rotating the fastener.

In an embodiment, the device is made of a solid material, in particular a polymer, biopolymer, copolymer, metal, or combinations thereof.

In an aspect, the present disclosure relates to an integration, assembly, surgical instrument or kit, comprising an eyelid speculum and the ophthalmic surgical device of any of the embodiments of the present disclosure, in particular the device and eyelid speculum being integrally formed.

### BRIEF DESCRIPTION OF THE DRAWINGS

The following figures provide preferred embodiments for illustrating the disclosure and should not be seen as limiting the scope of invention.

FIG. 1 shows a perspective view of an embodiment of the disclosed device for eye positioning and immobilization using traction suture or sutures.

FIG. 2 shows a top view of an embodiment of the disclosed device for eye positioning and immobilization using traction suture or sutures.

FIG. 3 shows the front lateral view of a patient having an embodiment of the of the disclosed device for eye positioning and immobilization using traction suture or sutures arranged below the eye and supported against a speculum-supported lower eyelid and/or the speculum itself.

FIG. 4 shows several views of an embodiment of the disclosed device for eye positioning and immobilization using traction suture or sutures with a rotatable fastener, with optional guiding slots in the laterally protruding tabs (FIG. 6B) or without (FIG. 6A).

FIG. 5 shows several views of an embodiment of the disclosed device for eye positioning and immobilization using traction suture or sutures without a rotatable fastener, with optional guiding slots in the laterally protruding tabs (FIG. 7B) or without (FIG. 7A).

### REFERENCE LIST

(1) Base of the ophthalmic surgical device for eye positioning and immobilization

(2) Anchoring element

(3) Suture deflector(s)

(4) Support edge

(5) Laterally protruding tabs

(6) Ridge

(7) Suture

(8) Speculum

(9) Rotatable fastener

(10) Sulcus

(11) Guiding slots

### DETAILED DESCRIPTION

The present disclosure relates to an eye positioning and immobilization device for use during ophthalmological surgery, in particular during glaucoma surgery.

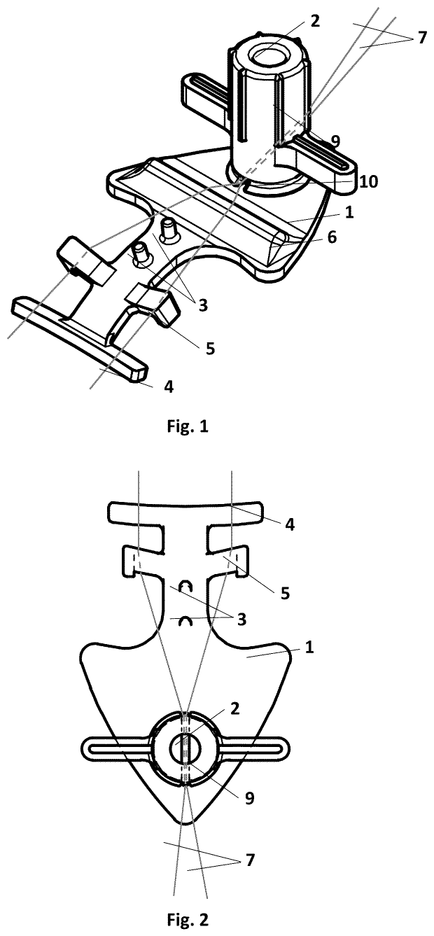

FIG. 1 shows a perspective view of an embodiment of the disclosed device for eye positioning and immobilization using traction suture or sutures. FIG. 2 shows a top view of an embodiment of the disclosed device for eye positioning and immobilization using traction suture or sutures.

In FIG. 1 and FIG. 2, it is disclosed an ophthalmic surgical device for eye positioning and immobilization using traction suture or sutures 7, comprising:
a base 1 configured for securing to a periocular region comprising the inferior periocular area and frontotemporal area, said base having an adhesive surface for adhering to the periocular region;
wherein the base comprises an anchoring element 2 for adjustable fixation [i.e. the fixation is a reversible so that adjustment can be provided] of the traction suture or sutures.

In an embodiment, the anchoring element 2 comprises a recess [for example, a slot or a slit 10] for the suture or sutures to pass through and for holding said suture or sutures.

In an embodiment, the anchoring element 2 comprises a rotatable fastener 9 for fastening the suture or sutures.

In an embodiment, the rotatable fastener 9 is rotatably coupled to the anchoring element 2 [i.e. the anchoring element serves as a rotation axle of the fastener].

In an embodiment, the rotatable fastener 9 comprises a sulcus 10 for the suture or sutures to wind around the rotatable fastener and for holding said suture or sutures.

In an embodiment, the rotatable fastener 9 comprises one or more recesses (or sulcus) for the suture or sutures to pass through and for holding said suture or sutures, in particular the one or more recesses (or sulcus) being arranged on a surface of the rotatable fastener facing said base.

In an embodiment, the anchoring element 2 comprises a clip-on fastener for fastening the suture or sutures.

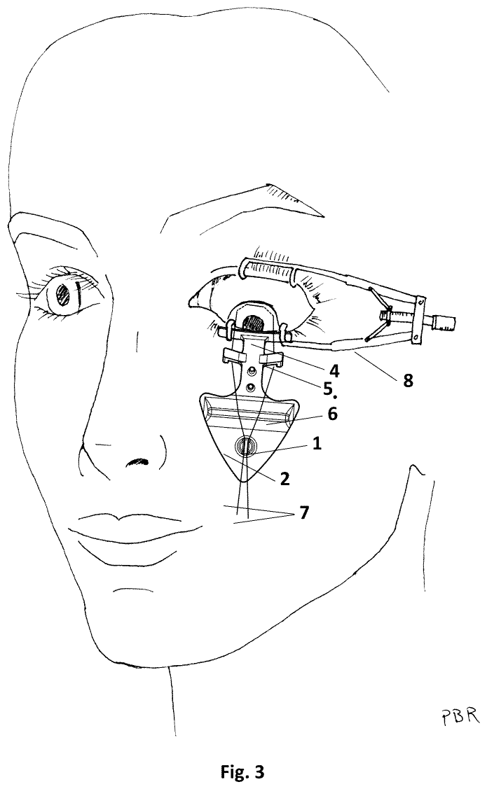

FIG. 3 shows the front-lateral view of a patient having an embodiment of the disclosed device for eye positioning and immobilization using traction suture or sutures arranged below the eye and supported against a speculum-supported lower eyelid and/or the speculum itself.

In FIGS. 1-5, the base has a support edge 4 for supporting against at least a part of a speculum-supported eyelid or against at least a part of an eyelid speculum.

In an embodiment, the base further comprises two laterally protruding tabs 5 arranged between the support edge and the anchoring element for guiding the one or more sutures towards the periocular area.

In an embodiment, the base further comprises a ridge 6 for applying friction to the suture or sutures, and raising the suture or sutures from the base towards a suture running position in the anchoring element or the fastener, if existing.

An embodiment further comprises one or more protrusions 3 between the support edge and the anchoring element for deflecting the one or more sutures, in particular the base comprising a plurality of said protrusions wherein the plurality of the protrusions are arranged along an axis between the support edge and the anchoring element.

In an embodiment, the base 1 has a curvature, or a curvature and countercurvature, for contouring the periocular area.

In an embodiment, the base 1 is an elongated base having two ends.

In an embodiment, the base 1 has a narrowing between first and second end.

In an embodiment, the base 1 has two laterally protruding tabs 5 arranged on the narrowed region of the base.

In an embodiment, the anchoring element 2 is a protruding anchor.

In an embodiment, the anchoring element 2 is an anchor with a hollow volume for passing suture or sutures through and anchoring the traction suture or sutures.

In an embodiment, the rotatable fastener 9 is a rotatable knob, rotatable torniquet or a rotatable screw cap.

In an embodiment, the rotatable fastener 9 comprises one or more levers for rotating the fastener.

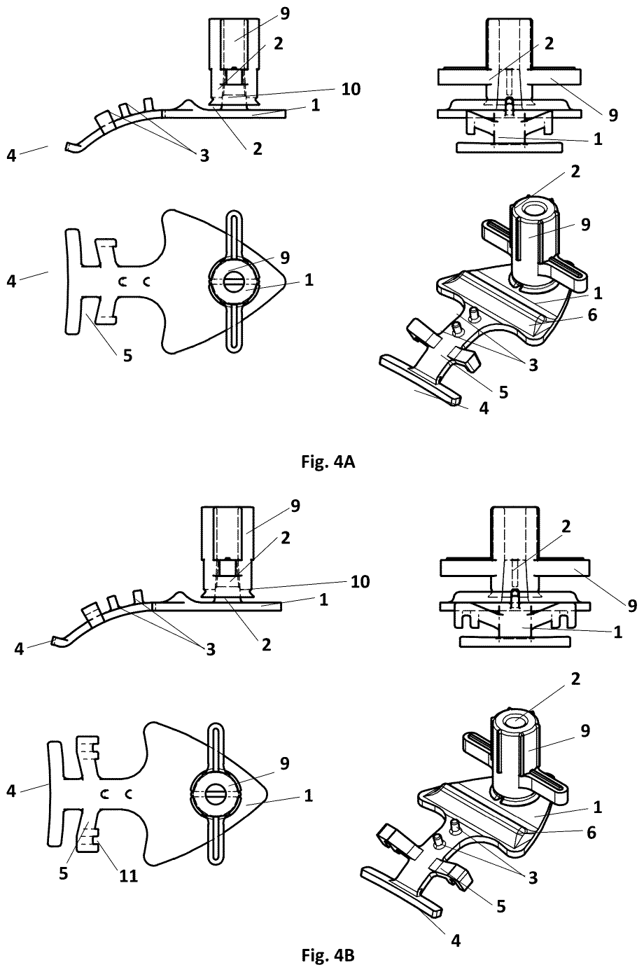

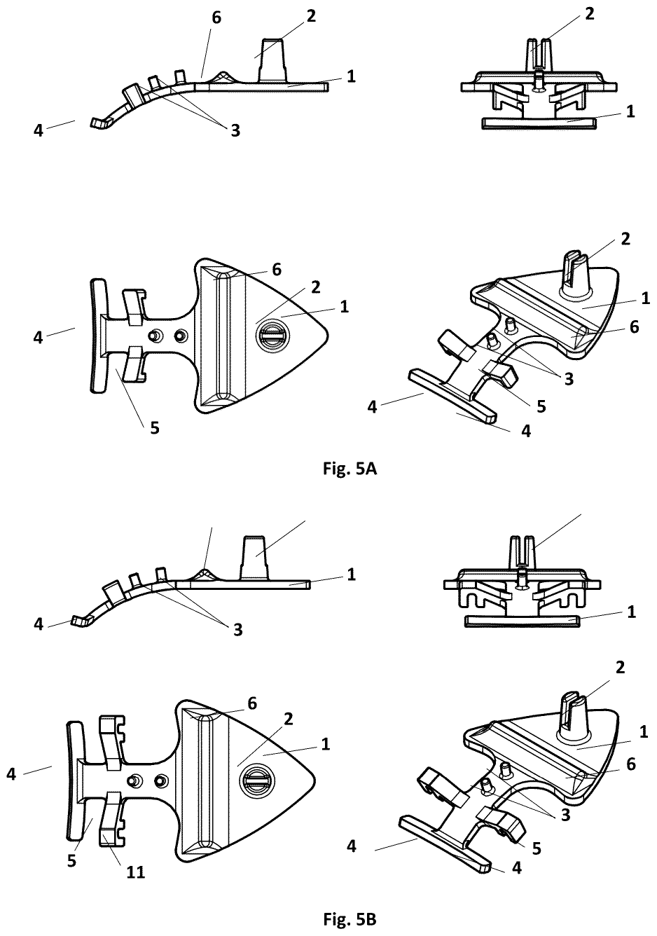

In FIG. 4 and FIG. 5, it is disclosed embodiments of the ophthalmic surgical device for eye positioning and immobilization using traction suture or sutures 7, with and without, respectively, a rotatable fastener. FIGS. 3 and 4 show optional guiding slots 11 in the laterally protruding tabs (FIGS. 4B, 5B) or without (FIGS. 4A, 5A).

The disclosed device can be made of a suitable solid material, a polymer, a biopolymer, a copolymer, metal, or combinations thereof. The embodiments described above are combinable.

1. Ophthalmic surgical device for eye globe positioning and immobilization using eye globe traction suture or sutures (7) and for use with an eyelid speculum (8), comprising:
a base (1) configured for securing to a periocular region comprising the inferior periocular and frontotemporal area, said base having an adhesive surface for adhering to the periocular region;
wherein the base comprises an anchoring element (2) for adjustable fixation of the traction suture or sutures;
wherein the anchoring element (2) is configured for receiving the eye globe traction suture or sutures (7) when the device is adhered to a periocular region by the adhesive surface;
wherein the base (1) has a support edge (4) for supporting, externally to the eyelids, against at least a part of a speculum-supported eyelid of the eye or against at least a part of an eyelid speculum (8); and
wherein the anchoring element (2) is located directly opposite, relative to the base (1), to a region comprised in the adhesive surface.

2. Ophthalmic surgical device for eye globe positioning and immobilization according to claim 1 wherein the base (1) further comprises two laterally protruding tabs (5) arranged between the support edge (4) and the anchoring element (2) for guiding the one or more sutures (7) in particular the two laterally protruding tabs (5) being arranged for guiding the one or more sutures (7) in the needed direction and towards the orbital rim.

3. (canceled)

4. Ophthalmic surgical device for eye globe positioning and immobilization according to claim 1 wherein the anchoring element (2) comprises a recess for the suture or sutures (7) to pass through and for holding said suture or sutures (7).

5. Ophthalmic surgical device for eye globe positioning and immobilization according to claim 1 wherein the anchoring element (2) comprises a rotatable fastener (9) for fastening the suture or sutures (7), in particular wherein the rotatable fastener (9) is rotatably coupled to the anchoring element (2).

6. Ophthalmic surgical device for eye globe positioning and immobilization according to claim 5 wherein the rotatable fastener (9) comprises a sulcus (10) for the suture or sutures (7) to wind around the rotatable fastener (9) and for holding said suture or sutures (7), in particular wherein the rotatable fastener (9) comprises one or more recesses for the suture or sutures (7) to pass through and for holding said suture or sutures (7).

7. Ophthalmic surgical device for eye globe positioning and immobilization according to claim 1 wherein the anchoring element comprises a detachable fastener for fastening the suture or sutures, in particular the detachable fastener also being a rotatable fastener.

8. Ophthalmic surgical device for eye globe positioning and immobilization according to claim 1 wherein the base (1) further comprises a ridge (6) for applying friction to the suture or sutures (7), and raising the suture or sutures (7) from the base (1) towards a suture running position in the anchoring element (2) or the fastener, if existing.

9. Ophthalmic surgical device for eye globe positioning and immobilization according to claim 1 the base (1) further comprises one or more protrusions (3) between the support edge (4), including the support edge itself, and the anchoring element (2) for deflecting the one or more sutures (7).

10. Ophthalmic surgical device for eye globe positioning and immobilization according to claim 1 wherein the base (1) has a curvature, or curvature and counter curvature for contouring adjacently the periocular area.

11. Ophthalmic surgery device for eye globe positioning and immobilization according to claim 1, wherein the base (1) is an elongated base having two ends, in particular the base having a narrowing between first and second end, further in particular wherein the base (1) further comprises two laterally protruding tabs (5) arranged on the narrowed region of the base (1) and arranged between the support edge (4) and the anchoring element (2) for guiding the one or more sutures (7).

12. (canceled)

13. (canceled)

14. Ophthalmic surgical device for eye globe positioning and immobilization according to claim 1 wherein the anchoring element (2) is a protruding anchor, in particular comprising a fastener (9) with a matching hollow volume for coupling with said protruding anchor to anchor said suture or sutures (7), in particular shaped to anchor at least two sutures fastened in opposite slip directions for reducing suture slippage.

15. (canceled)

16. Ophthalmic surgical device for eye globe positioning and immobilization according to claim 1 wherein the anchoring element (2) is an anchor with a hollow volume for passing through and anchoring the traction suture or sutures (7), in particular comprising a fastener with a matching protrusion for coupling with said hollow volume to anchor and lock said suture or sutures (7), further in particular shaped to anchor at least two sutures fastened in opposite slip directions for reducing suture slippage.

17. (canceled)

18. Ophthalmic surgical device for eye globe positioning and immobilization according to claim 1, wherein the anchoring element (2) is shaped to taper away from the base (1), for increasing grip when the fastener is pushed towards the base (1), in particular the anchoring element (2) being conical.

19. Ophthalmic surgical device for eye globe positioning and immobilization according to claim 5, wherein the rotatable fastener (9) is a rotatable knob, rotatable torniquet or a rotatable screw cap, and/or wherein the rotatable fastener comprises one or more levers for assisting on rotation of the fastener.

20. Ophthalmic surgical device for eye globe positioning and immobilization according to claim 1 wherein the device is made of a solid material, in particular a polymer, biopolymer, copolymer, metal, or combinations thereof.

21. Integration, assembly, surgical instrument or kit, comprising an eyelid speculum (8) and the ophthalmic surgical device according to claim 1.
